(19) United States
(12) Patent Application Publication
(10) Pub. No.: US 2020/0315474 A1
(43) Pub. Date: Oct. 8, 2020

(54) BIOSIGNAL SENSOR

(71) Applicant: Murata Manufacturing Co., Ltd., Nagaokakyo-shi (JP)

(72) Inventors: Tsutomu YAMASAKI, Nagaokakyo-shi (JP); Kaname HANADA, Nagaokakyo-shi (JP)

(21) Appl. No.: 16/908,777

(22) Filed: Jun. 23, 2020

Related U.S. Application Data
(63) Continuation of application No. PCT/JP2018/038791, filed on Oct. 18, 2018.

(30) Foreign Application Priority Data
Feb. 23, 2018 (JP) 2018-030714

(51) Int. Cl. A61B 5/024; H01L 31/02; H01L 31/0232; H01L 31/173

(52) U.S. Cl. CPC A61B 5/02427; H01L 31/02019; A61B 5/02433; H01L 31/173; H01L 31/02327

(57) ABSTRACT

A biosignal sensor includes a substrate, first light-emitting elements, second light-emitting elements, and light-receiving elements. The light-receiving elements are positioned individually spaced apart by different distances from the first light-emitting elements and the second light-emitting elements and receive light from the first light-emitting elements and the second light-emitting elements. The plurality of light-receiving elements are linearly or substantially linearly aligned in an X direction. The first light-emitting elements and the second light-emitting elements are located at positions different in a Y direction from positions of the plurality of light-receiving elements.

### CROSS REFERENCE TO RELATED APPLICATIONS

This application claims the benefit of priority to Japanese Patent Application No. 2018-030714 filed on Feb. 23, 2018 and is a Continuation Application of PCT Application No. PCT/JP2018/038791 filed on Oct. 18, 2018. The entire contents of each application are hereby incorporated herein by reference.

### BACKGROUND OF THE INVENTION

### 1. Field of the Invention

The present invention relates to a biosignal sensor that detects biosignals with light.

### 2. Description of the Related Art

A biosignal sensor composed of a light-emitting element and a light-receiving element mounted on a substrate is known (refer to, for example, Japanese Unexamined Patent Application Publication No. 2017-169690). The biosignal sensor described in Japanese Unexamined Patent Application Publication No. 2017-169690 includes a light-emitting element that is provided on a substrate and configured to emit light toward a subject under measurement and at least three light-receiving elements that are positioned on the substrate at different distances from the light-emitting element.

The biosignal sensor described in Japanese Unexamined Patent Application Publication No. 2017-169690 uses a single light-emitting element to detect biosignals. As a result, the strength of light from the light-emitting element may be insufficient and it may be impossible to detect biosignals of sufficient signal strength, so that the measurement may be unreliable. Since the distance from the light-emitting element differs among the at least three light-receiving elements, the measurement depth of the subject under measurement can be changed in accordance with the distances. However, because the single light-emitting element that emits light of a single wave length is used, in comparison to the case of using lights of a plurality of wave lengths, the amount of information of biosignals may be decreased and the signal-to-noise ratio (S/N) with respect to biosignals may be degraded. Additionally, since the light-emitting element and the at least three light-receiving elements are linearly aligned, the size of the sensor tends to increase with respect to the direction in which these elements are aligned.

### SUMMARY OF THE INVENTION

Preferred embodiments of the present invention provide biosignal sensors that each are able to significantly increase a signal-to-noise ratio (S/N) with respect to biosignals and also to provide biosignal sensors with a significantly reduced size.

A biosignal sensor of a preferred embodiment of the present invention includes a substrate that extends in an X direction and a Y direction perpendicular or substantially perpendicular to each other, a first light-emitting element that is provided on one major side surface of the substrate and that emits light of a first wave length toward a subject under measurement, a second light-emitting element that is provided on the one major side surface of the substrate at a position adjacent to or in a vicinity of the first light-emitting element and that emits light of a second wave length different from the first wave length toward the subject under measurement, and a plurality of light-receiving elements that are positioned on the one major side surface of the substrate individually spaced apart by different distances from the first light-emitting element and the second light-emitting element and that receive light from the first light-emitting element and the second light-emitting element. The plurality of light-receiving elements are linearly or substantially linearly aligned in the X direction. The first light-emitting element and the second light-emitting element are located at positions different in the Y direction from positions of the plurality of light-receiving elements.

The preferred embodiments of the present invention are able to significantly increase the S/N with respect to biosignals and significantly reduce a size of a biosignal sensor.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a perspective view of a biosignal sensor according to a first preferred embodiment of the present invention.

FIG. 2 is a plan view of the biosignal sensor.

FIG. 3 is a sectional view of the biosignal sensor when viewed in a direction indicated by arrows of in FIG. 2.

FIG. 4 is a bottom view of the biosignal sensor.

FIG. 5 is a block diagram showing internal features of an electronic component.

FIG. 6 shows reflected light from a living body in the case in which a first light-emitting element and a second light-emitting element are spaced apart from a light-receiving element by a shortest distance.

FIG. 7 shows reflected light from a living body in the case in which a first light-emitting element and a second light-emitting element are spaced apart from a light-receiving element by a distance between the shortest distance and a longest distance.

FIG. 8 shows reflected light from a living body in the case in which a first light-emitting element and a second light-emitting element are spaced apart from a light-receiving element by the longest distance.

FIG. 9 is a plan view of a biosignal sensor according to a second preferred embodiment of the present invention.

FIG. 10 is a sectional view of the biosignal sensor when viewed in a direction indicated by arrows of X-X in FIG. 9.

FIG. 11 is a plan view of a biosignal sensor according to a third preferred embodiment of the present invention.

FIG. 12 is a sectional view of the biosignal sensor when viewed in a direction indicated by arrows of XII-XII in FIG. 11.

### DETAILED DESCRIPTION OF THE PREFERRED EMBODIMENTS

Hereinafter, biosignal sensors according to preferred embodiments of the present invention will be described with reference to the accompanying drawings.

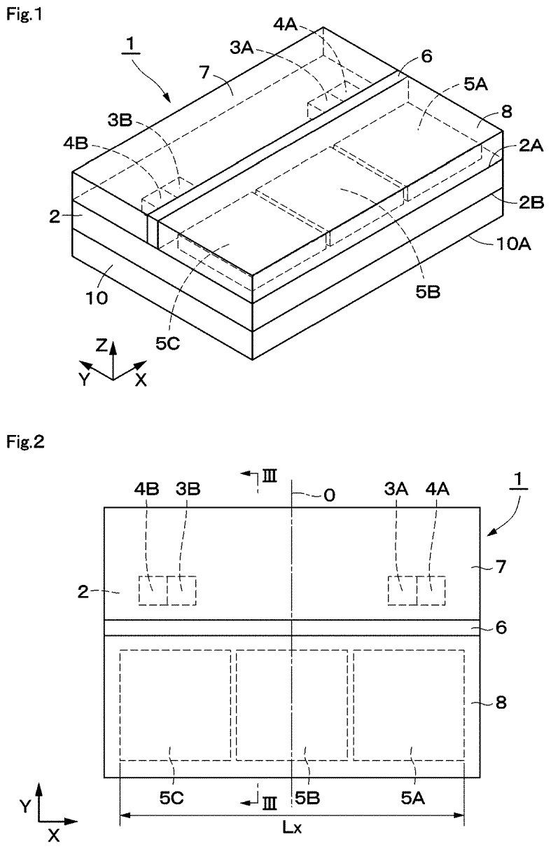

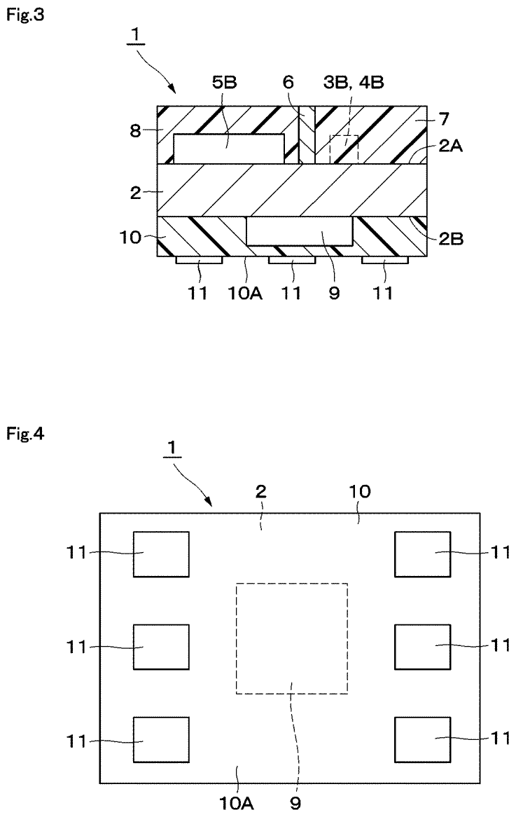

FIGS. 1 to 4 show a biosignal sensor 1 according to a first preferred embodiment of the present invention. The biosignal sensor 1 detects, for example, photoplethysmographic signals (pulse wave signals) representing pulse on a living body as a subject under measurement. The biosignal sensor 1 includes a substrate 2, first light-emitting elements 3A and 3B, second light-emitting elements 4A and 4B, light-receiving elements 5A to 5C, and the like.

The substrate 2 is a flat plate made of an insulating material. The substrate 2 extends in an X direction and a Y direction that are perpendicular or substantially perpendicular to each other. Here, the width direction of the substrate 2 is a Z direction perpendicular or substantially perpendicular to the X direction and the Y direction. For example, a printed circuit board or a ceramic board is included as the substrate 2. The substrate 2 may be a multilayer substrate formed by laminating a plurality of electrode layers and insulating layers. The plurality of electrode layers and insulating layers are alternated with one another. On a front surface 2A (one major side surface) of the substrate 2, the first light-emitting elements 3A and 3B, the second light-emitting elements 4A and 4B, and the light-receiving elements 5A to 5C are mounted as optical components. On a back surface 2B (the other major side surface) of the substrate 2, an electronic component 9 is mounted. As such, the substrate 2 is a double-sided mounting substrate. On the front surface 2A of the substrate 2, only the optical components (the first light-emitting elements 3A and 3B, the second light-emitting elements 4A and 4B, and the light-receiving elements 5A to 5C) of the optical components and the electronic component 9 are mounted.

The first light-emitting elements 3A and 3B are preferably defined by, for example, light-emitting diodes (LEDs), laser diodes (LDs), vertical-cavity surface-emitting lasers (VCSELs), or cavity LEDs. The first light-emitting elements 3A and 3B emit, for example, red light or infrared light in a band of about 600 nm to about 1000 nm as a light L1 of a first wave length. The first light-emitting elements 3A and 3B are affixed to the front surface 2A of the substrate 2 by a bonding method, for example, die bonding or wire bonding. The first light-emitting elements 3A and 3B are aligned in the X direction. The first light-emitting elements 3A and 3B are spaced apart from each other in the X direction. The first light-emitting elements 3A and 3B are electrically coupled to the electronic component 9.

Similarly to the first light-emitting elements 3A and 3B, the second light-emitting elements 4A and 4B are preferably defined by, for example, LEDs or the like. The second light-emitting elements 4A and 4B emit, for example, green light in a band of about 495 nm to about 570 nm as a light L2 of a second wave length different from the first wave length. Accordingly, the second light-emitting elements 4A and 4B emit the light L2 of a wave length shorter than the wave lengths of the light L1 of the first light-emitting elements 3A and 3B. The second light-emitting elements 4A and 4B are affixed to the front surface 2A of the substrate 2 by a bonding method, for example, die bonding or wire bonding. The second light-emitting elements 4A and 4B are electrically coupled to the electronic component 9.

The light-receiving elements 5A to 5C are preferably defined by, for example, photodiodes (PDs) or the like. The light-receiving elements 5A to 5C are located at positions different from the positions of the first light-emitting elements 3A and 3B and the second light-emitting elements 4A and 4B on the front surface 2A (the one major side surface) of the substrate 2. The light-receiving elements 5A to 5C are electrically coupled to the electronic component 9.

The three light-receiving elements 5A to 5C are located at positions spaced apart by different distances from the first light-emitting elements 3A and 3B and the second light-emitting elements 4A and 4B on the front surface 2A of the substrate 2. Thus, the distance between the first light-emitting element 3A and the light-receiving element 5A, the distance between the first light-emitting element 3A and the light-receiving element 5B, and the distance between the first light-emitting element 3A and the light-receiving element 5C are different from each other. Here, the distance between the first light-emitting element 3A and the light-receiving element 5A is the shortest. The distance between the first light-emitting element 3A and the light-receiving element 5C is the longest. The distance between the first light-emitting element 3A and the light-receiving element 5B is an in-between length.

Furthermore, the distance between the first light-emitting element 3B and the light-receiving element 5A, the distance between the first light-emitting element 3B and the light-receiving element 5B, and the distance between the first light-emitting element 3B and the light-receiving element 5C are different from each other. Here, the distance between the first light-emitting element 3B and the light-receiving element 5A is the longest. The distance between the first light-emitting element 3B and the light-receiving element 5C is the shortest. The distance between the first light-emitting element 3B and the light-receiving element 5B is an in-between length. Similarly, the distance to the second light-emitting element 4A differs among the light-receiving elements 5A to 5C. The distance to the second light-emitting element 4B differs among the light-receiving elements 5A to 5C. The three light-receiving elements 5A to 5C are aligned in the X direction.

The three light-receiving elements 5A to 5C receive the lights L1 and L2 from the first light-emitting elements 3A and 3B and the second light-emitting elements 4A and 4B. The light-receiving elements 5A to 5C convert (photoelectric conversion) received optical signals into, for example, electrical signals such as current signals and output the electrical signals. Specifically, the light-receiving elements 5A to 5C receive the lights L1 and L2 that have been emitted by the first light-emitting elements 3A and 3B and the second light-emitting elements 4A and 4B and have been reflected by a living body and convert the received lights L1 and L2 into detection signals S as electrical signals. The detection signal S corresponding to the first light-emitting elements 3A and 3B and the detection signal S corresponding to the second light-emitting elements 4A and 4B may be separated from each other. The light-receiving elements 5A to 5C outputs the detection signal S to the electronic component 9. The light-receiving elements 5A to 5C are affixed to the front surface 2A of the substrate 2 by employing a bonding method, for example, die bonding or wire bonding. The light-receiving elements 5A to 5C may be defined by, for example, phototransistors.

The first light-emitting elements 3A and 3B are located at positions different from the positions of the three light-receiving elements 5A to 5C in the Y direction. Similarly, the second light-emitting elements 4A and 4B are located at positions different from the positions of the three light-receiving elements 5A to 5C in the Y direction. The second light-emitting element 4A is positioned adjacent to or in a vicinity of the first light-emitting element 3A. The second light-emitting element 4B is positioned adjacent to or in a vicinity of the first light-emitting element 3B. The first light-emitting elements 3A and 3B and the second light-emitting elements 4A and 4B are offset on one side (the upper side in FIG. 2) in the Y direction with respect to the three light-receiving elements 5A to 5C.

The three light-receiving elements 5A to 5C are positioned to spread across a range of the predetermined length dimension Lx in the X direction. The first light-emitting elements 3A and 3B and the second light-emitting elements 4A and 4B are positioned in the X direction within the range of the predetermined length dimension Lx in which the three light-receiving elements 5A to 5C are located. The two first light-emitting elements 3A and 3B are positioned on both end sides in the X direction in which the three light-receiving elements 5A to 5C are aligned. The two second light-emitting elements 4A and 4B are positioned on both end sides in the X direction in which the three light-receiving elements 5A to 5C are aligned. Specifically, when the first light-emitting element 3A and the second light-emitting element 4A, and the light-receiving element 5A are located at the same or substantially the same position in the Y direction, the first light-emitting element 3A and the second light-emitting element 4A are positioned to overlap the light-receiving element 5A. When the first light-emitting element 3B and the second light-emitting element 4B, and the light-receiving element 5C are located at the same or substantially the same position in the Y direction, the first light-emitting element 3B and the second light-emitting element 4B are positioned to overlap the light-receiving element 5C.

The two first light-emitting elements 3A and 3B are positioned symmetrically or substantially symmetrically across a central axis O perpendicular or substantially perpendicular to a straight line along which the three light-receiving elements 5A to 5C are aligned. The two second light-emitting elements 4A and 4B are positioned symmetrically or substantially symmetrically across the central axis O perpendicular or substantially perpendicular to the straight line along which the three light-receiving elements 5A to 5C are aligned. The two second light-emitting elements 4A and 4B are positioned at outer side portions in the X direction with respect to the two first light-emitting elements 3A and 3B. The two second light-emitting elements 4A and 4B may be positioned at inner side portions in the X direction with respect to the two first light-emitting elements 3A and 3B or may be offset in the Y direction.

A wall 6 is provided on the front surface 2A side of the substrate 2 and blocks light between the first light-emitting elements 3A and 3B and the second light-emitting elements 4A and 4B, and the light-receiving elements 5A to 5C. The wall 6 is preferably made of a resin material in a non-transparent color, for example, black, to block the lights L1 and L2 from the first light-emitting elements 3A and 3B and the second light-emitting elements 4A and 4B.

The wall 6 extends in the X direction in parallel or substantially parallel to the light-receiving elements 5A to 5C that are provided in line. The wall 6 is positioned, with respect to the Y direction, between the first light-emitting elements 3A and 3B and the second light-emitting elements 4A and 4B, and the light-receiving elements 5A to 5C. The wall 6 significantly reduces or prevents the lights L1 and L2 from the first light-emitting elements 3A and 3B and the second light-emitting elements 4A and 4B from directly striking the light-receiving elements 5A to 5C.

The first light-emitting elements 3A and 3B and the second light-emitting elements 4A and 4B are overlaid with a transparent resin component or element 7. The light-receiving elements 5A to 5C are overlaid with a transparent resin component or element 8. The transparent resin components or elements 7 and 8 are preferably made of a resin material (a transparent resin material), for example, that the lights L1 and L2 from the first light-emitting elements 3A and 3B and the second light-emitting elements 4A and 4B or reflected light from a subject under measurement are able to pass through.

The transparent resin components or elements 7 and 8 are provided on the front surface 2A of the substrate 2 by potting or transfer molding, for example.

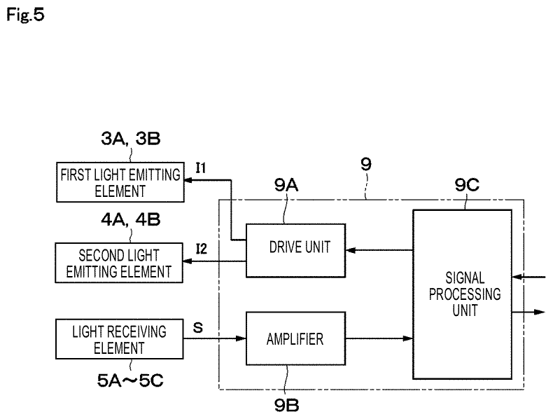

The electronic component 9 is defined by an integrated circuit component (an IC component), for example. As shown in FIG. 5, the electronic component 9 includes, for example, an actuator 9A, an amplifier 9B, and a signal processor 9C. The electronic component 9 is mounted on the back surface 2B (the other major side surface) of the substrate 2 and positioned to overlap the first light-emitting elements 3A and 3B, the second light-emitting elements 4A and 4B, and the light-receiving elements 5A to 5C. Thus, the electronic component 9, and the first light-emitting elements 3A and 3B, the second light-emitting elements 4A and 4B, and the light-receiving elements 5A to 5C are layered in a height direction (the width direction of the substrate 2) with the substrate 2 provided therebetween.

An input side of the actuator 9A is coupled to the signal processor 9C. An output side of the actuator 9A is coupled to the first light-emitting elements 3A and 3B and the second light-emitting elements 4A and 4B. The actuator 9A supplies drive currents I1 and I2 to the first light-emitting elements 3A and 3B and the second light-emitting elements 4A and 4B in accordance with drive signals from the signal processor 9C. The drive currents I1 and I2 are subjected to pulse modulation at, for example, a predetermined frequency in accordance with drive signals from the signal processor 9C. As a result, the first light-emitting elements 3A and 3B and the second light-emitting elements 4A and 4B flash on and off. Times at which light is emitted differ between the first light-emitting elements 3A and 3B and the second light-emitting elements 4A and 4B.

An input side of the amplifier 9B is coupled to the light-receiving elements 5A to 5C. An output side of the amplifier 9B is coupled to the signal processor 9C. The amplifier 9B is preferably, for example, a transimpedance amplifier (TIA) and converts the detection signals S as current signals from the light-receiving elements 5A to 5C into voltage signals and amplifies the voltage signals. A filter to de-noise may be provided between the amplifier 9B and the signal processor 9C.

An output side of the signal processor 9C is coupled to the actuator 9A. An input side of the signal processor 9C is coupled to the amplifier 9B. Additionally, the signal processor 9C is coupled to the outside via a mount substrate (not shown in the drawing).

The signal processor 9C includes, for example, a DA converter (DAC) and an AD converter (ADC). The signal processor 9C converts drive signals as digital signals inputted from the outside into analog signals by the DA converter. The signal processor 9C converts the detection signals S as analog signals inputted from the light-receiving element 4 via the amplifier 9B into digital signals by the AD converter. The electronic component 9 is not necessarily a single component. Hence, for example, the actuator 9A, the amplifier 9B, the signal processor 9C may be individual electronic components.

A base 10 is provided on the back surface 2B side of the substrate 2 and covers the electronic component 9.

The base 10 is preferably made of, for example, a resin insulating material. The base 10 includes a back surface 10A (a bottom surface) defining a flat plane. A plurality of electrode terminals 11 are provided on the back surface 10A. To form the base 10, a resin material having fluidity is applied to cover the electronic component 9 in a state in which the electronic component 9 and conductive pins are affixed to the back surface 2B of the substrate 2. The resin material is then cured, and the base 10 is formed.

The electrode terminals 11 are provided at the back surface 10A of the base 10, and are exposed. The electrode terminals 11 are electrically coupled to, for example, the signal processor 9C of the electronic component 9. Specifically, for example, the conductive pins made of a conductive metal as columnar conductors are affixed to the back surface 2B of the substrate 2. Base end sides of the conductive pins are affixed to the substrate and are electrically coupled to the electronic component 9. Front end surfaces of the conductive pins are exposed at the back surface 10A of the base 10 and define the electrode terminals 11. As a result, the electrode terminals 11 input drive signals from the outside to the signal processor 9C and output detection signals from the signal processor 9C to the outside.

The biosignal sensor 1 according to the first preferred embodiment of the present invention has the structure described above, and the operation thereof will be described below.

The biosignal sensor 1 is a surface mount device including the electrode terminals 11 provided at the back surface 10A of the base 10. The biosignal sensor 1 is mounted at a front surface of a mount substrate (not shown in the drawing) including electrodes provided at the front surface. The electrode terminals 11 of the biosignal sensor 1 are joined to the electrodes of the mount substrate. As a result, the electronic component 9 of the biosignal sensor 1 is coupled to an external processing circuit formed at the mount substrate.

The electronic component 9 supplies the drive currents I1 and I2 to the first light-emitting elements 3A and 3B and the second light-emitting elements 4A and 4B in accordance with drive signals from the external processing circuit. The first light-emitting elements 3A and 3B and the second light-emitting elements 4A and 4B send the lights L1 and L2 to a living body as a subject under measurement in accordance with the drive currents I1 and I2. The light-receiving elements 5A to 5C receive reflected lights that are based on the lights L1 and L2 and that are reflected by the living body and in turn output the detection signals S. The electronic component 9 converts the detection signals S into digital signals and outputs the digital signals to the external processing circuit.

The reflected light from a living body is attenuated depending on the level of hemoglobin concentration. As a result, the external processing circuit is able to extract photoplethysmographic signals corresponding to pulse of the living body based on the detection signals S based on reflected light.

The three light-receiving elements 5A to 5C are located at positions spaced apart by different distances from the first light-emitting elements 3A and 3B and the second light-emitting elements 4A and 4B. Accordingly, the biosignal sensor 1 includes the three light-receiving elements 5A to 5C for one light source (any of the first light-emitting elements 3A and 3B and the second light-emitting elements 4A and 4B).

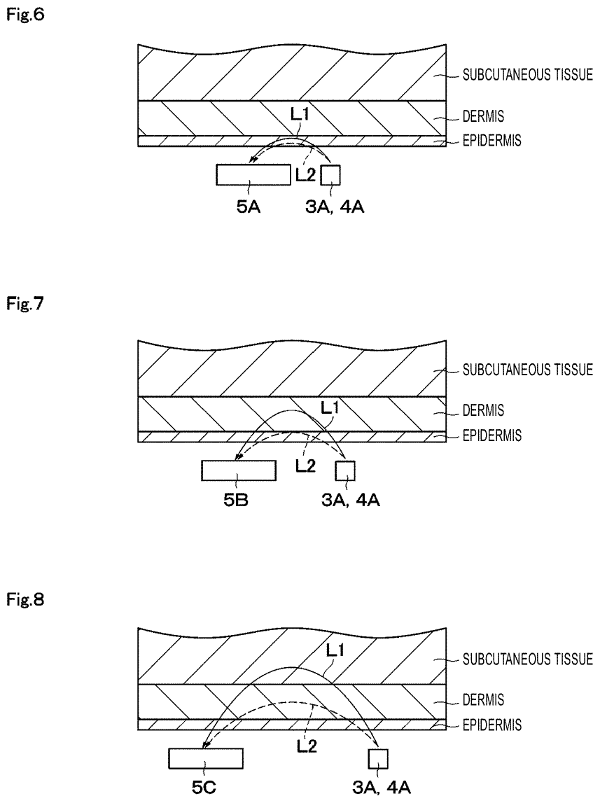

As shown in FIGS. 6 to 8, for example, when the one first light-emitting element 3A defining and functioning as a light source is brought into focus, the light-receiving element 5A is positioned spaced apart by a shortest distance from the first light-emitting element 3A. The light-receiving element 5C is positioned spaced apart by a longest distance from the first light-emitting element 3A. The light-receiving element 5B is positioned spaced apart by an in-between distance from the first light-emitting element 3A with respect to the light-receiving element 5A and the light-receiving element 5C. Accordingly, with respect to the light-receiving element 5A, the measurement depth of living tissue is relatively small; and the light-receiving element 5A receives, for example, biosignals adjacent to or in a vicinity of the epidermis. In the case of the light-receiving element 5C, the measurement depth of living tissue is relatively large; and the light-receiving element 5C receives, for example, biosignals adjacent to or in a vicinity of the subcutaneous tissue. In the case of the light-receiving element 5B, the measurement depth of living tissue is between the measurement depth of the light-receiving element 5A and the measurement depth of the light-receiving element 5C; and the light-receiving element 5B receives, for example, biosignals adjacent to or in a vicinity of the dermis. Similarly, when the second light-emitting element 4B is brought into focus, the measurement depth of living tissue increases in the following order: the light-receiving element 5A, the light-receiving element 5B, and the light-receiving element 5C. When the first light-emitting element 3B and the second light-emitting element 4B are brought into focus, the measurement depth of living tissue decreases in the following order: the light-receiving element 5A, the light-receiving element 5B, and the light-receiving element 5C. As described above, the measurement depth of living tissue differs among the light-receiving elements 5A to 5C.

As a result, when biosignals are measured with respect to a plurality of subjects under measurement, biosignals are able to be detected at measurement depths that are detection positions suitable for the respective subjects under measurement by the light-receiving elements 5A to 5C. Therefore, the signal-to-noise ratio (S/N) with respect to biosignals is significantly increased. In particular, since the measurement depth of living tissue differs among the light-receiving elements 5A to 5C, light is able to be targeted at the dermis, at which noise is relatively less, while the subcutaneous tissue, which includes much fat defining and functioning as a cause of noise, is avoided, by selecting any of the light-receiving elements 5A to 5C.

Furthermore, the biosignal sensor 1 includes, as the same-color light sources, the two first light-emitting elements 3A and 3B, and the two second light-emitting elements 4A and 4B. As a result, the strength of light emission and the measurement area of living body are able to be both increased, and thus, biosignals are able to be measured with increased reliability in comparison to the case in which weak light is applied to a minute range. In addition, with respect to the first light-emitting elements 3A and 3B and the second light-emitting elements 4A and 4B, the emitted lights L1 and L2 differ from each other in the wave length, and as a result, the measurement depth of living tissue is able to be changed between the light L1 from the first light-emitting elements 3A and 3B and the light L2 from the second light-emitting elements 4A and 4B. Thus, according to the lights L1 and L2 of two kinds of wave lengths, biosignals are able to be detected from portions at different measurement depths, so that biosignals are able to be detected at appropriate detection positions.

As described above, in the biosignal sensor 1 according to the first preferred embodiment, the three light-receiving elements 5A to 5C are aligned in the X direction while the two first light-emitting elements 3A and 3B and the two second light-emitting elements 4A and 4B are located at positions different from the positions of the three light-receiving elements 5A to 5C in the Y direction.

Accordingly, the three light-receiving elements 5A to 5C are positioned individually spaced apart by different distances from the first light-emitting elements 3A and 3B and the second light-emitting elements 4A and 4B and receive the lights L1 and L2 from the first light-emitting elements 3A and 3B and the second light-emitting elements 4A and 4B. As a result, the measurement depth of a subject under measurement is able to be changed with respect to each of the light-receiving elements 5A to 5C in accordance with the distance from the first light-emitting elements 3A and 3B and the second light-emitting elements 4A and 4B. In addition, since the first light-emitting elements 3A and 3B and the second light-emitting elements 4A and 4B that respectively emit lights of different wave lengths are provided, the measurement depth of a subject under measurement is able to be changed depending on the wave length of light. As a result, biosignals of a subject under measurement are able to be detected at predetermined measurement depths, so that the S/N with respect to biosignals is significantly increased.

Furthermore, since the two first light-emitting elements 3A and 3B and the two second light-emitting elements 4A and 4B are provided at the substrate 2, in comparison to the case of including a single light-emitting element, the strength of light emission and the measurement area of subject under measurement are able to be both increased, and thus, reliable measurement is able to be provided. Moreover, the first light-emitting elements 3A and 3B and the second light-emitting elements 4A and 4B are located at positions different from the positions of the three light-receiving elements 5A to 5C in the Y direction. Thus, the first light-emitting elements 3A and 3B and the second light-emitting elements 4A and 4B, and the three light-receiving elements 5A to 5C are provided not linearly but in parallel or substantially in parallel with each other. As a result, the external dimension of the biosignal sensor 1 in the X direction is able to be significantly reduced.

The three light-receiving elements 5A to 5C are positioned to spread across the range of the predetermined length dimension Lx in the X direction, while the first light-emitting elements 3A and 3B and the second light-emitting elements 4A and 4B are positioned in the X direction within the range of the predetermined length dimension Lx in which the three light-receiving elements 5A to 5C are located. Accordingly, in comparison to the case in which the first light-emitting elements 3A and 3B and the second light-emitting elements 4A and 4B, and the three light-receiving elements 5A to 5C are linearly or substantially linearly provided, the length dimension of the biosignal sensor 1 in the X direction be is able to be significantly reduced, and as a result, the biosignal sensor 1 is able to be significantly reduced in size. In addition, the biosignal sensor 1 is highly sensitive while the biosignal sensor 1 is significantly reduced in size in one package. As a result, design flexibility for the mount substrate is able to be increased.

The two first light-emitting elements 3A and 3B are positioned on both end sides in the X direction in which the three light-receiving elements 5A to 5C are aligned, while the two second light-emitting elements 4A and 4B are positioned on both sides in the X direction in which the three light-receiving elements 5A to 5C are aligned. Accordingly, light is able to be applied from the first light-emitting elements 3A and 3B and the second light-emitting elements 4A and 4B to a subject under measurement from both sides in the X direction. Thus, light is able to be applied from the two first light-emitting elements 3A and 3B to a portion of a subject under measurement situated between the two first light-emitting elements 3A and 3B. Similarly, light is able to be applied from the two second light-emitting elements 4A and 4B to a portion of a subject under measurement situated between the two second light-emitting elements 4A and 4B. Consequently, biosignals of a subject under measurement are able to be detected by strong light emitted by the two first light-emitting elements 3A and 3B and the two second light-emitting elements 4A and 4B, and as a result, the S/N with respect to biosignals is able to be significantly increased.

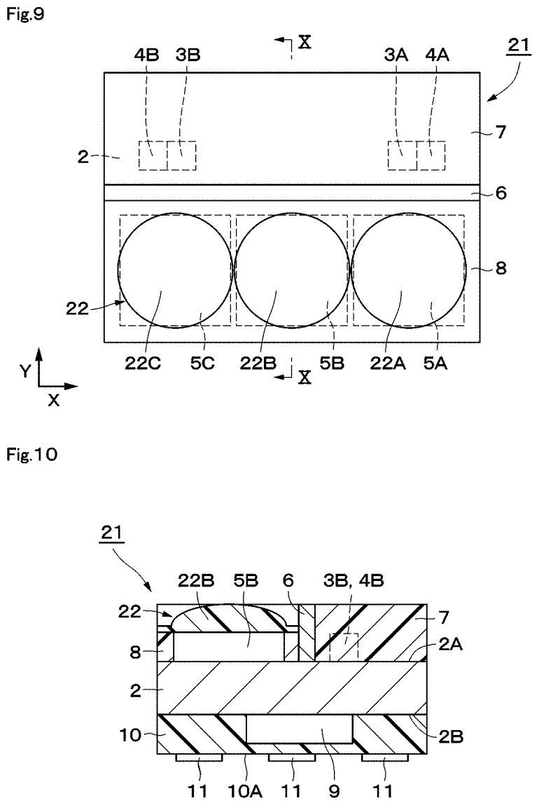

Next, a second preferred embodiment of the present invention is described with reference to FIGS. 9 and 10. In the second preferred embodiment, three light-receiving elements are overlaid with a lens array including three or more lenses positioned to respectively face the light-receiving elements. In the second preferred embodiment, components and elements the same as or similar to those of the first preferred embodiment are assigned the same reference characters and description thereof is omitted.

A biosignal sensor 21 according to the second preferred embodiment includes, similarly to the first preferred embodiment, the substrate 2, the first light-emitting elements 3A and 3B, the second light-emitting elements 4A and 4B, the light-receiving elements 5A to 5C, and the like. In addition to these, in the biosignal sensor 21, the three light-receiving elements 5A to 5C are overlaid with a lens array 22 including three lenses 22A to 22C positioned to respectively face the light-receiving elements 5A to 5C. The lens array 22 includes a transparent resin material and is provided as optical components differently from the light-receiving elements 5A to 5C. Thus, the lens array 22 is attached to light-receiving surface sides of the light-receiving elements 5A to 5C by a joining process, for example, as bonding.

The second preferred embodiment as described above is able to provide effects and advantages the same as or similar to those of the first preferred embodiment described above. The three light-receiving elements 5A to 5C are overlaid with the lens array 22. Accordingly, when a subject under measurement reflects light emitted by the first light-emitting elements 3A and 3B and the second light-emitting elements 4A and 4B, the lenses 22A to 22C of the lens array 22 are able to concentrate the reflected light on the light-receiving elements 5A to 5C. Thus, in comparison to the case without a lens array, the responsivity is able to be increased and the S/N with respect to biosignals is able to be further increased. In addition, since the lens array 22, which is an optical element, is provided in the biosignal sensor 21, when the biosignal sensor 21 is attached to a mount substrate, it is unnecessary to additionally install an optical component at the mount substrate, so that assembly operation is able to be simplified.

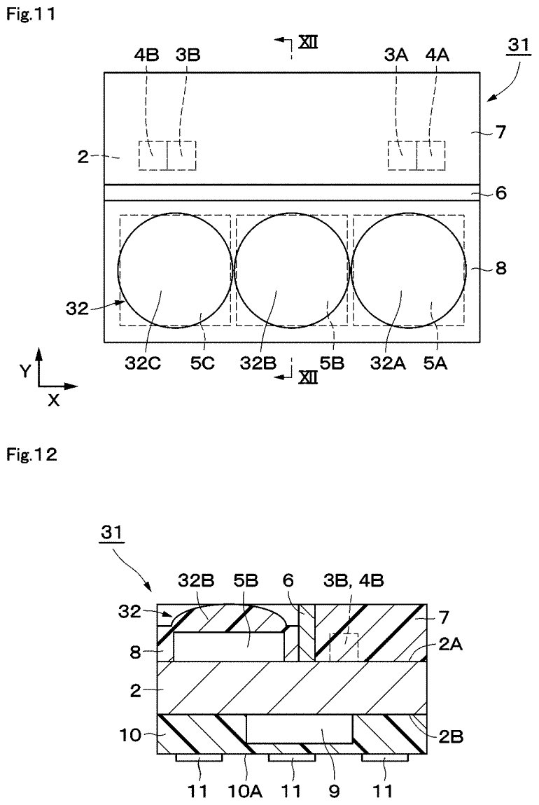

Next, a third preferred embodiment of the present invention is described with reference to FIGS. 11 and 12. In the third preferred embodiment, a lens array is integrated with light-receiving elements. In the third preferred embodiment, components and elements the same as or similar to those of the first preferred embodiment are assigned the same reference characters and description thereof is omitted.

A biosignal sensor 31 according to the third preferred embodiment includes, similarly to the first preferred embodiment, the substrate 2, the first light-emitting elements 3A and 3B, the second light-emitting elements 4A and 4B, the light-receiving elements 5A to 5C, and the like. In addition to these, in the biosignal sensor 31, the three light-receiving elements 5A to 5C are overlaid with a lens array 32 including three lenses 32A to 32C positioned to respectively face the light-receiving elements 5A to 5C. The lens array 32 is provided together with the transparent resin component or element 8 when the transparent resin component or element 8 is formed by, for example, transfer molding. As such, the lens array 32 is integrated with the light-receiving elements 5A to 5C.

The third preferred embodiment as described above is able to provide effects and advantages the same as or similar to those of the first preferred embodiment described above. Moreover, since the lens array 32 is integrated with the light-receiving elements 5A to 5C, the accuracy of positioning of the lens array 32 is able to be increased with the light-receiving elements 5A to 5C.

It should be noted that, while in the preferred embodiments described above, the biosignal sensors 1, 21, and 31 each include the two first light-emitting elements 3A and 3B and the two second light-emitting elements 4A and 4B, the present invention is not limited to this example and the biosignal sensor may include a single first light-emitting element and a single second light-emitting element, or three or more first light-emitting elements and three or more second light-emitting elements. Additionally, the biosignal sensor may include a third light-emitting element that emits light of a wave length different from the wave length of the first light-emitting elements 3A and 3B and the wave length of the second light-emitting elements 4A and 4B. While in the preferred embodiments described above the biosignal sensor includes the three light-receiving elements 5A to 5C, the biosignal sensor may include two light-receiving elements, or four or more light-receiving elements.

While in the preferred embodiments described above the two first light-emitting elements 3A and 3B and the two second light-emitting elements 4A and 4B are all located on one side in the Y direction with respect to the three light-receiving elements 5A to 5C aligned, the present invention is not limited to this example and the first light-emitting elements and the second light-emitting elements may be located on both sides in the Y direction with respect to the three light-receiving elements aligned.

While in the preferred embodiments described above the wave length of light emitted by the first light-emitting elements 3A and 3B is longer than the wave length of light emitted by the second light-emitting elements 4A and 4B, the present invention is not limited to this example and the wave length of light emitted by the first light-emitting element may be shorter than the wave length of light emitted by the second light-emitting element.

Further, the specific numerical values presented in the preferred embodiments described above are mere one example and the numerical values used as the example should not be construed in a limiting sense. These numerical values may be set in accordance with, for example, properties of an application target.

The preferred embodiments described above are mere examples, and the features described in the different preferred embodiments may be partially replaced or combined with each other.

Next, the invention embodied in the preferred embodiments described above is described. The biosignal sensor of the present invention includes a substrate that extends in the X direction and the Y direction perpendicular or substantially perpendicular to each other, the first light-emitting element that is provided on one major side surface of the substrate and that emits light of a first wave length toward a subject under measurement, the second light-emitting element that is provided on the one major side surface of the substrate at a position adjacent to or in a vicinity of the first light-emitting element and that emits light of a second wave length different from the first wave length toward the subject under measurement, and the plurality of light-receiving elements that are positioned on the one major side surface of the substrate individually spaced apart by different distances from the first light-emitting element and the second light-emitting element and that receive light from the first light-emitting element and the second light-emitting element. The plurality of light-receiving elements are aligned in the X direction. The first light-emitting element and the second light-emitting element are located at positions different in the Y direction from positions of the plurality of light-receiving elements.

Accordingly, the plurality of light-receiving elements are positioned individually spaced apart by different distances from the first light-emitting element and the second light-emitting element and receive light from the first light-emitting element and the second light-emitting element. As a result, the measurement depth of a subject under measurement is able to be changed with respect to each of the light-receiving elements in accordance with the distance from the first light-emitting element and the second light-emitting element. In addition, since the first light-emitting element and the second light-emitting element that respectively emit lights of different wave lengths are provided, the measurement depth of a subject under measurement is able to be changed depending on the wave length of light. As a result, biosignals of a subject under measurement are able to be detected at predetermined measurement depths, so that the S/N with respect to signals is significantly increased. Moreover, the first light-emitting element and the second light-emitting element are located at positions different from the positions of the plurality of light-receiving elements in the Y direction. Thus, the first light-emitting element and the second light-emitting element, and the plurality of light-receiving elements are provided not linearly but in parallel or substantially in parallel with each other. As a result, the external dimension of the biosignal sensor in the X direction is able to be significantly reduced.

In the preferred embodiments of the present invention, a plurality of the first light-emitting elements and a plurality of the second light-emitting elements are provided at the substrate. Thus, in comparison to the case of including a single light-emitting element, the strength of light emission and the measurement area of subject under measurement are able to be both increased, and thus, reliable measurement is able to be provided.

In the preferred embodiments of the present invention, the plurality of light-receiving elements are positioned to spread across the range of the predetermined length dimension in the X direction, while the first light-emitting elements and the second light-emitting elements are positioned in the X direction within the range of the predetermined length dimension in which the plurality of light-receiving elements are located. Accordingly, in comparison to the case in which the first light-emitting elements and the second light-emitting elements, and the plurality of light-receiving elements are linearly or substantially linearly provided, the length dimension of the biosignal sensor in the X direction is able to be significantly reduced, and as a result, the biosignal sensor is able to be significantly reduced in size.

In the preferred embodiments of the present invention, the first light-emitting elements are positioned separately on both end sides in the X direction in which the plurality of light-receiving elements are aligned and the second light-emitting elements are positioned separately on both end sides in the X direction in which the plurality of light-receiving elements are aligned. Accordingly, light from the first light-emitting elements and the second light-emitting elements is able to be applied to a subject under measurement from both sides in the X direction. Consequently, biosignals of a subject under measurement are able to be detected by strong light emitted by the plurality of first light-emitting elements and the plurality of second light-emitting elements.

In the preferred embodiments of the present invention, the plurality of light-receiving elements are overlaid with the lens array including the plurality of lenses positioned to respectively face the plurality of light-receiving elements. Accordingly, when a subject under measurement reflects light emitted by the first light-emitting element and the second light-emitting element, the lenses of the lens array are able to concentrate the reflected light on the light-receiving elements. Thus, in comparison to the case without the lens array, the responsivity is able to be increased.

In the preferred embodiments of the present invention, the lens array is integrated with the light-receiving elements. Accordingly, the accuracy of positioning of the lens array with the light-receiving elements is able to be significantly increased.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A biosignal sensor comprising:
a substrate extending in an X direction and a Y direction perpendicular or substantially perpendicular to each other;
a first light-emitting element provided on one major side surface of the substrate and that emits light of a first wave length toward a subject under measurement;
a second light-emitting element provided on the one major side surface of the substrate at a position adjacent to or in a vicinity of the first light-emitting element and that emits light of a second wave length different from the first wave length toward the subject under measurement; and
a plurality of light-receiving elements positioned on the one major side surface of the substrate individually spaced apart by different distances from the first light-emitting element and the second light-emitting element and that receive light from the first light-emitting element and the second light-emitting element; wherein
the plurality of light-receiving elements are linearly or substantially linearly aligned in the X direction; and
the first light-emitting element and the second light-emitting element are located at positions different in the Y direction from positions of the plurality of light-receiving elements.

2. The biosignal sensor according to claim 1, wherein a plurality of first light-emitting elements, each being the first light-emitting element, and a plurality of second light-emitting elements, each being the second light-emitting element, are provided at the substrate.

3. The biosignal sensor according to claim 2, wherein
the plurality of light-receiving elements are positioned to spread across a range of a predetermined length dimension in the X direction; and
the first light-emitting elements and the second light-emitting elements are positioned in the X direction within the range of the predetermined length dimension in which the plurality of light-receiving elements are located.

4. The biosignal sensor according to claim 3, wherein
the first light-emitting elements are positioned separately on both end sides of the substrate in the X direction in which the plurality of light-receiving elements are aligned; and
the second light-emitting elements are positioned separately on both end sides of the substrate in the X direction in which the plurality of light-receiving elements are aligned.

5. The biosignal sensor according to claim 1, wherein the plurality of light-receiving elements are overlaid with a lens array including a plurality of lenses positioned to respectively face the plurality of light-receiving elements.

6. The biosignal sensor according to claim 5, wherein the lens array is integrated with the light-receiving elements.

7. The biosignal sensor according to claim 1, wherein the substrate is a printed circuit board or a ceramic board.

8. The biosignal sensor according to claim 1, wherein the first light-emitting element and the second light-emitting element include at least one of light-emitting diodes (LEDs), laser diodes (LDs), vertical-cavity surface-emitting lasers (VCSELs), and cavity LEDs.

9. The biosignal sensor according to claim 1, wherein
the first light-emitting element emits red light or infrared light in a band of about 600 nm to about 1000 nm; and
the second light-emitting element emits green light in a band of about 495 nm to about 570 nm.

10. The biosignal sensor according to claim 1, wherein the first light-emitting element emits light having a higher wavelength than a wavelength of light emitted from the second light-emitting element.

11. The biosignal sensor according to claim 1, further comprising:
an electric component provided on a second major side surface of the substrate; wherein
each of the plurality of light-receiving elements is electrically coupled to the electronic component; and
each of the plurality of light-receiving elements outputs a detection signal to the electronic component.

12. The biosignal sensor according to claim 11, wherein the electronic component includes an actuator that outputs a first drive current to the first light-emitting element and a second drive current to the second light-emitting element, an amplifier that receives the detection signal of each of the plurality of light-receiving elements, and a signal processor.

13. The biosignal sensor according to claim 12, wherein the signal processor applies pulse modulation at a predetermined frequency to the first drive current and the second drive current.

14. The biosignal sensor according to claim 1, wherein at least one of the plurality of light-receiving elements is a photodiode.
